(19) United States
(12) Patent Application Publication
(10) Pub. No.: US 2026/0065507 A1
(43) Pub. Date: Mar. 5, 2026

(54) METHOD AND SYSTEM FOR DISTRACTION ALERT

(71) Applicant: MiTAC Digital Technology Corporation, Taoyuan City (TW)

(72) Inventor: Yu-Sheng CHANG, Taoyuan City (TW)

(21) Appl. No.: 19/310,069

(22) Filed: Aug. 26, 2025

(30) Foreign Application Priority Data
Aug. 27, 2024 (TW) 113132167

(51) Int. Cl. G06T 7/73; A61B 5/00; A61B 5/11; A61B 5/18; B60Q 9/00; G06V 20/59; G06V 40/10

(52) U.S. Cl. CPC G06T 7/75; A61B 5/0077; A61B 5/1114; A61B 5/1121; A61B 5/1128; A61B 5/18; A61B 5/6893; A61B 5/746; B60Q 9/00; G06V 20/597; G06T 2207/10016; G06T 2207/20081; G06T 2207/20084; G06T 2207/30196; G06T 2207/30268; G06V 40/103

(57) ABSTRACT

A method for distraction alert is implemented by a system disposed on a vehicle, the method includes: obtaining a video recording that contains a body portion and a head portion of a human body in real time; obtaining a body orientation of the body portion and a head orientation of the head portion based on the video recording; comparing the body orientation and the head orientation, so as to obtain a value of a relative angle between the body orientation and the head orientation; determining whether the value of the relative angle is greater than a predetermined threshold; and in response to determining that the value of the relative angle is greater than the predetermined threshold, outputting an alert message.

### CROSS-REFERENCE TO RELATED APPLICATION

This application claims priority to Taiwanese Invention Patent Application No. 113132167, filed on Aug. 27, 2024, the entire disclosure of which is incorporated by reference herein.

### FIELD

The disclosure relates to an alert system, and more particularly to a method and a system for distraction alert.

### BACKGROUND

To assist a driver of a vehicle to remain focused, a conventional alert system is configured to send out alert messages when detecting that the driver is distracted in an attempt to reduce the risk of traffic accidents caused by the driver being distracted. However, if the conventional alert system is overly sensitive, the alert messages may be output even when the driver is not distracted. In such a case, the conventional alert system not only fails to achieve its intended function, but also distracts the driver.

### SUMMARY

Therefore, an object of the disclosure is to provide a method and a system for distraction alert that can alleviate at least one of the drawbacks of the prior art.

According to an aspect of the disclosure, a method for distraction alert is to be implemented by a system that is disposed on a vehicle. The method includes: obtaining a video recording that contains a part of a human body in real time, where the part of the human body includes a body portion and a head portion; obtaining a body orientation of the body portion and a head orientation of the head portion based on the video recording; comparing the body orientation and the head orientation, so as to obtain a value of a relative angle between the body orientation and the head orientation; determining whether the value of the relative angle is greater than a predetermined threshold; and in response to determining that the value of the relative angle is greater than the predetermined threshold, outputting an alert message.

According to another aspect of the disclosure, a system for distraction alert is adapted to be disposed in a vehicle. The system includes a processing unit and an output unit that is electrically connected to the processing unit. The processing unit is configured to obtain a video recording that contains a part of a human body in real time, where the part of the human body includes a body portion and a head portion, and to obtain a body orientation of the body portion and a head orientation of the head portion based on the video recording. The processing unit is further configured to compare the body orientation and the head orientation, so as to obtain a value of a relative angle between the body orientation and the head orientation, and to determine whether the value of the relative angle is greater than a predetermined threshold. The processing unit is further configured to, in response to determining that the value of the relative angle is greater than the predetermined threshold, control the output unit to output an alert message.

### BRIEF DESCRIPTION OF THE DRAWINGS

Other features and advantages of the disclosure will become apparent in the following detailed description of the embodiment(s) with reference to the accompanying drawings. It is noted that various features may not be drawn to scale.

FIG. 1 is a block diagram illustrating a system for distraction alert according to an embodiment of the disclosure.

FIG. 2 is a schematic diagram illustrating 3-dimensional (3D) human model data that is stored in the system according to an embodiment of the disclosure.

FIG. 3 is a flow chart illustrating a method for distraction alert according to an embodiment of the disclosure.

FIG. 4 is a schematic diagram illustrating a video recording used in the method according to an embodiment of the disclosure.

### DETAILED DESCRIPTION

Before the disclosure is described in greater detail, it should be noted that where considered appropriate, reference numerals or terminal portions of reference numerals have been repeated among the figures to indicate corresponding or analogous elements, which may optionally have similar characteristics.

Throughout the disclosure, the term “connected to” may refer to a direct connection among a plurality of pieces of electrical apparatus/devices/equipment via an electrically conductive material (e.g., an electrical wire, a semi-conductive material), or an indirect connection between two pieces of electrical apparatus/devices/equipment via another one or more pieces of apparatus/devices/equipment, or wireless communication (e.g., Wi-Fi, Bluetooth®, electromagnetic conduction).

Throughout the disclosure, the term “unit” refers to computer hardware rather than software. For example, a “processing unit” is used to represent computer hardware with data processing capabilities. Additionally, the term “unit” may refer to a single computer hardware with a specific function, or a group of computer hardware with similar functions. For example, a “processing unit” may refer to a single processor with data processing functions, but may also refer to a collection of processors.

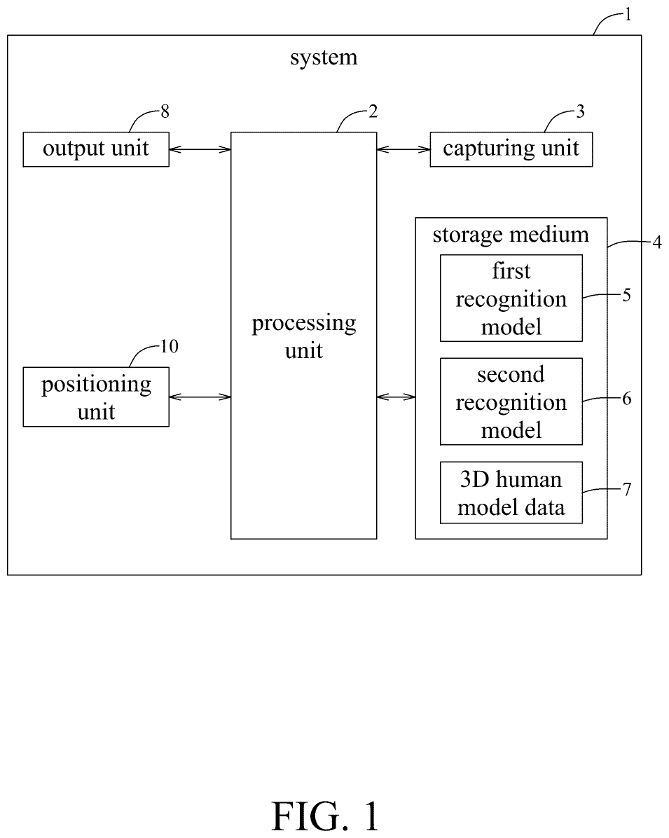

Referring to FIG. 1, according to an embodiment of the disclosure, a system 1 for distraction alert is adapted to be disposed in a vehicle (not shown). In one embodiment, the vehicle is, for example, a large cargo truck. In other embodiments, the vehicle may be other types of large vehicles, small trucks, sedans, or sport utility vehicles (SUVs).

In this embodiment, the system 1 is implemented by a digital video recorder (DVR) that has image recognition and alert outputting functions, such as a driving recorder, but the disclosure is not limited to such. In this embodiment, the system 1 may be independently manufactured and sold, and may be mounted onto the vehicle after the vehicle has been manufactured. In some embodiments, the system 1 is not limited to a DVR, and may be mounted onto the vehicle as a built-in safety assistance system of the vehicle when the vehicle is being manufactured.

The system 1 includes a processing unit 2, and a capturing unit 3, a storage medium 4 and an output unit 8 that are electrically connected to the processing unit 2.

In this embodiment, the processing unit 2 may be implemented by a central processing unit (CPU) that has data computing and processing functions. The storage medium 4 may be embodied using one or more computer-readable storage mediums such as hard disk drives or flash memory. The capturing unit 3 may be a video camera that includes a lens and a light sensing component (not shown) and that is configured to record videos. The capturing unit 3 is disposed in the vehicle to record a video of a driver of the vehicle (e.g., the capturing unit 3 may be disposed in the front portion of the vehicle and faces the driver's seat). In this embodiment, the output unit 8 includes a speaker that is adapted to be mounted in the vicinity of the driver's seat. In some embodiments, the output unit 8 may further include a display, a light indicator module (e.g., a light emitting diode (LED)), a vibrator (e.g., disposed in the steering wheel), or combinations thereof. In some embodiments, the output unit 8 may include the vibrator without the speaker.

In one embodiment, to ensure that the processing unit 2 is able to receive videos obtained by the capturing unit 3 as soon as possible, the processing unit 2, the capturing unit 3 and the storage medium 4 are all disposed in the vehicle, and the processing unit 2 has wired connection to the capturing unit 3 and the storage medium 4. However, in some embodiments, the storage medium 4 may be implemented by a cloud server connected to the processing unit 2 through a network (e.g., the Internet), and the processing unit 2 may be wirelessly connected to the capturing unit 3 using wireless communication technology. In some embodiments, the capturing unit 3 is not included in the system 1. To describe in further detail, the processing unit 2 may be configured to be electrically connected to an external video recording module. That is to say, the capturing unit 3 is an external video recording module electrically connected to the processing unit 2, and is not part of the system 1. It should be noted that implementation of the processing unit 2, the capturing unit 3, the storage medium 4 and the output unit 8 described in this embodiment is merely an example, and the disclosure is not limited to such.

In this embodiment, the storage medium 4 stores a first recognition model 5, a second recognition model 6, and 3-dimensional (3D) human model data 7. Specifically, both the first recognition model 5 and the second recognition model 6 are realized using machine learning technology, and may be loaded and executed by the processing unit 2.

In this embodiment, the first recognition model 5 is a head recognition model that is configured to identify features of a head portion including eyes, ears, nose and mouth from a human body. In one example, the first recognition model 5 is implemented using a practical facial landmark detector (PFLD), but the disclosure is not limited to such.

To describe in further detail, the first recognition model 5 is trained with a first set of human body images as its training materials using deep learning. Each of the first set of human body images is a picture that contains a head portion of a human body. Specifically, the first set of human body images includes images of head portions of human bodies viewed from different angles (e.g., front view, side view, or front diagonal view), so that during training of the first recognition model 5, the first recognition model 5 may learn the features of the head portions viewed from different angles. In one example, the features of the head portions mentioned above may include, but are not limited to, shapes of eyes, ears, nose and mouth viewed from different angles, and relative positions among the eyes, ears, nose and mouth. As such, after the first recognition model 5 has been trained with deep learning based on the first set of human body images, the processing unit 2 may use the first recognition model 5 to identify the features of a head portion from an image or a video clip (e.g., a section of a video recording) that contains a head portion of a human body.

In this embodiment, the second recognition model 6 is an upper body recognition model that is configured to identify an upper body portion and a head portion from a human body. In one example, the second recognition model 6 is implemented using a high-resolution network (HRNet): a deep high-resolution representation learning for human pose estimation, but the disclosure is not limited to such.

To describe in further detail, the second recognition model 6 is trained with a second set of human body images as its training materials using deep learning. Each of the second set of human body images is a picture that contains at least an upper body portion of a human body. Specifically, the second set of human body images includes images of upper body portions of human bodies viewed from different angles (e.g., front view, side view, rear view, front diagonal view, and rear diagonal view), so that during training of the second recognition model 6, the second recognition model 6 may learn features of an upper body portion viewed from different angles. In one example, the features of an upper body portion mentioned above may include, but are not limited to, shapes of left arm, right arm, left shoulder, right shoulder, eyes, ears, nose and mouth viewed from different angles (e.g., front view and front diagonal view), and relative positions among them. As such, after the second recognition model 6 has been trained with deep learning based on the second set of human body images, the processing unit 2 may use the second recognition model 6 to identify features of an upper body portion from an image or a video clip (e.g., a section of a video recording) that contains an upper body portion of a human body.

It should be noted that the first set of human body images used for training the first recognition model 5 may be completely identical to, partially identical to, or completely different from the second set of human body images used for training the second recognition model 6. Moreover, using machine learning to achieve computer recognition of specific targets (such as the aforementioned features of the head portion and the upper body portion) can be achieved using existing technologies, and thus the training process of the first recognition model and the second recognition model 6 will not be described in further detail for the sake of brevity.

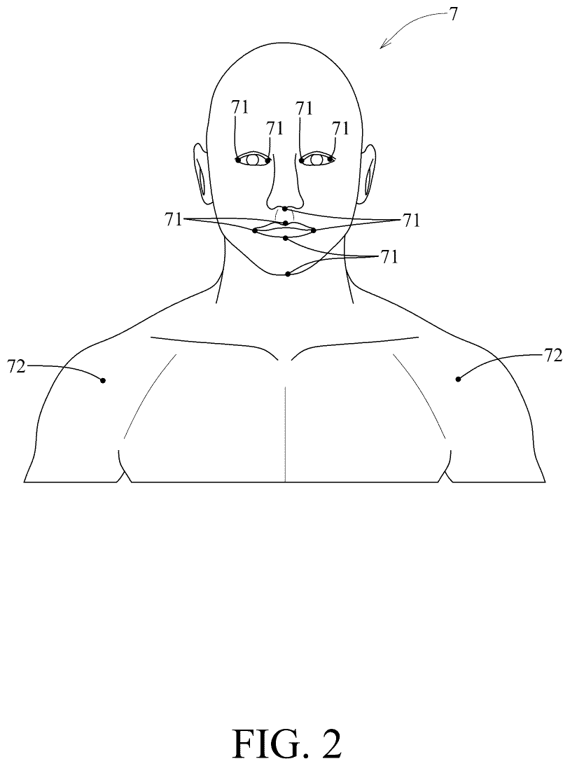

The 3D human model data 7 is a 3D human body model, and as shown in FIG. 2, the 3D human model data 7 may be visualized on a display screen in a form of a 3D virtual human body. The 3D human model data 7 includes a plurality of reference feature points that are located on the virtual human body and that respectively correspond to a plurality of reference coordinate sets. The reference coordinate sets are based on a virtual 3D coordinate system, and each of the reference coordinate sets is one 3D coordinate set that indicates a position of the corresponding reference feature point on the 3D human body model in the virtual 3D coordinate system. In this embodiment, the reference feature points includes ten reference head feature points 71 and two reference shoulder feature points 72 as exemplified in FIG. 2.

Referring further to FIGS. 2 to 4, a method for distraction alert according to an embodiment of the disclosure is implemented by the system 1 while a driver is driving the vehicle. Prior to performing the method, the processing unit 2 makes a pose of the virtual human body in the virtual 3D coordinate system serve as a standard pose (i.e., a standard pose of a head portion and a standard pose of a body portion).

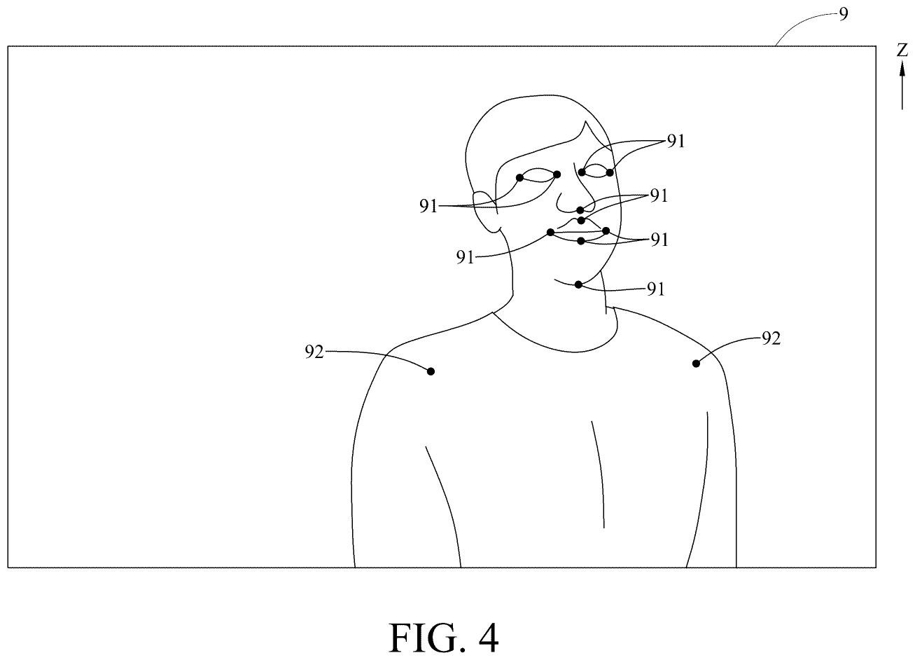

In step S31, the processing unit 2 controls the capturing unit 3 to continuously record videos to obtain a video recording 9 (as exemplified in FIG. 4) in real time from the capturing unit 3. The video recording 9 contains a part of a human body (i.e., the body of the driver, including a body portion and a head portion). Specifically, the video recording 9 is a real-time recording that is generated by the capturing unit 3. Once the processing unit 2 receives the video recording 9, the flow of the method proceeds to step S32.

In step S32, the processing unit 2 loads the first recognition model and the second recognition model 6 from the storage medium 4, and analyzes the video recording 9 using the first recognition model 5 and the second recognition model 6, so as to identify a plurality of determined feature points on the body of the driver in the video recording 9. The determined feature points are respectively associated with the reference feature points in the 3D human model data 7, and respectively correspond to a plurality of determined coordinate sets that are based on a 2-dimensional (2D) coordinate system. Each of the determined coordinate sets is one 2D coordinate set that indicates a position of the corresponding determined feature point in the video recording 9 in the 2D coordinate system.

To describe in further detail, as exemplified in FIG. 4, the determined feature points in this embodiment includes ten determined head feature points 91 (located at the head portion) and two determined shoulder feature points 92 (respectively located at two shoulder areas of the body portion). The determined head feature points 91 are respectively associated with the reference head feature points 71, and the determined shoulder feature points 92 are respectively associated with the reference shoulder feature points 72. Specifically, the determined head feature points 91 are identified by the processing unit 2 using the first recognition model 5, and the determined shoulder feature points 92 are identified by the processing unit 2 using the second recognition model 6.

Once the processing unit 2 identifies the determined head feature points 91 and the determined shoulder feature points 92 in the video recording 9, the flow proceeds to step S33.

In step S33, the processing unit 2 obtains head conversion data and body conversion data using a perspective-n-point pose algorithm based on the 3D human model data 7 and the determined coordinate sets that correspond respectively to the determined feature points. Each of the head conversion data and the body conversion data is a rotation matrix, which may be represented by a symbol of “^{c}T_{W}” in a mathematical expression for the perspective-n-point pose algorithm.

To describe in further detail, since the determined feature points are respectively associated with the reference feature points in the 3D human model data 7, the determined coordinate sets that correspond respectively to the determined feature points are also respectively associated with the reference coordinate sets that correspond respectively to the reference feature points. In this embodiment, the head conversion data is obtained by the processing unit 2 using the perspective-n-point pose algorithm based on ten reference head coordinate sets (i.e., ten of the reference coordinate sets) that correspond respectively to the ten reference head feature points 71 (as shown in FIG. 2), and based on ten determined head coordinate sets (i.e., ten of the determined coordinate sets) that correspond respectively to the ten determined head feature points 91 (as shown in FIG. 4). The head conversion data represents a coordinate conversion relationship between the ten determined head coordinate sets (in the 2D coordinate system) that correspond respectively to the ten determined head feature points 91 and the ten reference head coordinate sets (in the virtual 3D coordinate system) that correspond respectively to the ten reference head feature points 71. On the other hand, the body conversion data is obtained by the processing unit 2 using the perspective-n-point pose algorithm based on the reference coordinate sets that correspond respectively to the reference feature points (i.e., the ten reference head feature points 71 and the two reference shoulder feature points 72 as exemplified in FIG. 2), and based on the determined coordinate sets that correspond respectively to the determined feature points (i.e., the ten determined head feature points 91 and the two determined shoulder feature points 92 as exemplified in FIG. 4). The body conversion data represents a coordinate conversion relationship between the determined coordinate sets (in the 2D coordinate system) that correspond respectively to the determined feature points and the reference coordinate sets (in the virtual 3D coordinate system) that correspond respectively to the reference feature points.

It should be noted that the perspective-n-point pose algorithm is an existing mathematical method (reference may be made to E. Marchand et al., “Pose Estimation for Augmented Reality: A Hands-On Survey,” IEEE Transactions on Visualization and Computer Graphics, 2016, 22 (12), pp. 2633-2651. 10.1109/TVCG.2015.2513408. hal-01246370), and step S33 will not be described in further detail for the sake of brevity.

Once the processing unit 2 obtains the head conversion data and the body conversion data, the flow proceeds to step S34.

In step S34, the processing unit 2 obtains a head orientation of the head portion based on the head conversion data, and obtains a body orientation of the body portion based on the body conversion data.

In this embodiment, the head orientation indicates a first angle difference of a current pose of the head portion of the human body displayed in the video recording 9 relative to the standard pose of the head portion of the virtual human body with respect to a z-axis (i.e., the yaw axis). In one example, the head orientation may indicate that the current pose of the head portion is rotated by 20 degrees in the clockwise direction with respect to the z-axis relative to the standard pose of the head portion. The body orientation indicates a second angle difference of a current pose of the body portion of the human body displayed in the video recording 9 relative to the standard pose of the body portion of the virtual human body with respect to the z-axis. In one example, the body orientation may indicate that the current pose of the body portion is rotated by 35 degrees in the clockwise direction with respect to the z-axis relative to the standard pose of the body portion.

In this embodiment, the processing unit 2 obtains the head orientation as an Euler angle between the current pose of the head portion and the standard pose of the head portion with respect to the z-axis based on the head conversion data (that is a rotation matrix in this embodiment). Similarly, the processing unit 2 obtains the body orientation as an Euler angle between the current pose of the body portion and the standard pose of the body portion with respect to the z-axis based on the body conversion data (that is a rotation matrix in this embodiment). It should be noted that the calculation of an Euler angle is an existing mathematical method (see “Computing Euler angles from a rotation matrix” by Gregory G. Slabaugh for reference), and step S34 will not be described in further detail for the sake of brevity.

Once the processing unit 2 obtains the head orientation and the body orientation, the flow proceeds to step S35.

In step S35, the processing unit 2 compares the body orientation and the head orientation, so as to obtain a value of a relative angle between the body orientation and the head orientation. In this embodiment, the relative angle is a difference in angle between the body orientation and the head orientation with respect to the z-axis, that is, an angle at which the head portion is deflected to the right or left relative to the body portion. Once the processing unit 2 obtains the relative angle, the flow proceeds to step S36.

In step S36, the processing unit 2 determines whether the value of the relative angle is greater than a predetermined threshold (e.g., 10 degrees). That is to say, the processing unit 2 determines whether the current pose of the head portion is deflected to the left or to the right relative to the current pose of the body portion by an angle which has a value that is more than the predetermined threshold. In this embodiment, the relative angle is used as a base for determining whether the driver is distracted. When the determination in step S36 is affirmative, the flow proceeds to step S37; otherwise, the flow proceeds to step S38.

In step S37, in response to determining that the value of the relative angle is greater than the predetermined threshold, the processing unit 2 controls the output unit 8 to output an alert message so as to warn the driver. In this embodiment, the alert message is a sound emitted by the speaker, and a volume of the sound is gradually increased if the driver remains distracted (i.e., if a duration of the driver being distracted continues to increase). That is, the longer the driver is distracted, the louder the sound outputted by the speaker is. In some embodiments where the output unit 8 further includes the vibrator in the steering wheel, processing unit 2 may further control the vibrator in the steering wheel to vibrate, so as to enhance the effect of warning the driver.

In step S38, in response to determining that the value of the relative angle is not greater than the predetermined threshold, the processing unit 2 does not control the output unit 8 to output the alert message. That is to say, the processing unit 2 only outputs the alert message when determining that the value of the relative angle is greater than the predetermined threshold, thereby ensuring that the driver will not be surprised or be affected by the alert message when the driver is not actually distracted, which further ensures safety for driving.

In some embodiments, in addition to determining the relative angle with respect to the z-axis (i.e., the yaw axis), another relative angle between the head portion and the body portion with respect to a pitch axis may also be taken into consideration when determining whether the predetermined threshold has been exceeded.

In some embodiments, the system 1 further includes a positioning unit 10 that is electrically connected to the processing unit 2. The positioning unit may be implemented by a Global Positioning System (GPS) device that is configured to obtain a current position of the vehicle in a longitude-latitude coordinate system or in a map navigation coordinate system that is related to a digital navigation map which is stored in the processing unit 2. The positioning unit is configured to obtain location data related to the current position of the vehicle in real time, and send the location data to the processing unit 2 for the processing unit 2 to determine whether the vehicle is located in a pausing area based on the location data. In one example, the pausing area is a T-intersection or a crossroad that requires the driver to check the surroundings. When the processing unit 2 determines that the vehicle is located in the pausing area, the processing unit 2 generates and outputs a pausing notification to the output unit 8 for the output unit 8 to not output the alert message even if the value of the relative angle is greater than the predetermined threshold. This ensures that when the driver is driving in the pausing area, which may require the driver to turn his/her head or body to check the surroundings, no alert message is output by the output unit 8 to distract the driver. In one embodiment, the location data is a coordinate set of the location of the vehicle, where the processing unit 2 determines whether the vehicle is located in the pausing area based on the coordinate set of the location of the vehicle and the digital navigation map that is stored in the processing unit 2. In one example, when the processing unit 2 determines that the location data indicates that the vehicle is less than a predetermined distance (e.g., 200 meters) from a crossroad, the processing unit 2 determines that the vehicle is in the pausing area.

It should be noted that, when driving a large vehicle, the driver often needs to make large movements, such as turning the whole body to check the rearview mirror or observe the surroundings. Additionally, since the space of the driver's seat of a large vehicle is relatively large (compared to small vehicles), when the vehicle is making a turn, the driver is more likely to turn and tilt the entire upper body (including head and torso). That is to say, if the alert message is output solely based on the head orientation, the alert message may be output even when the driver is not actually distracted. Therefore, by calculating the relative angle between the body orientation and the head orientation for determining whether to output the alert message based on the predetermined threshold, the embodiment ensures that the alert message is output only when the relative angle is greater than the predetermined threshold, thus preventing the alert message from distracting the driver when unnecessary.

In this embodiment, the determined feature points are not limited to be ten determined head feature points 91 and two determined shoulder feature points 92. Moreover, the determined head feature points 91 and the determined shoulder feature points 92 may be identified through manual labelling, or may be identified through non-manual labelling (e.g., as in step S32 mentioned above). It should be noted that a quantity of the determined head feature points 91, a quantity of the determined shoulder feature points 92, and a method for identifying the determined feature points are not limited to the abovementioned example.

In this embodiment, the system 1 stores the first recognition model and the second recognition model 6 for performing the method; however, the disclosure should not be limited to such. In some embodiments, the system 1 may store a convolutional neural network (CNN) to replace the first recognition model 5 and the second recognition model 6 for performing the method while still achieving the function of the method.

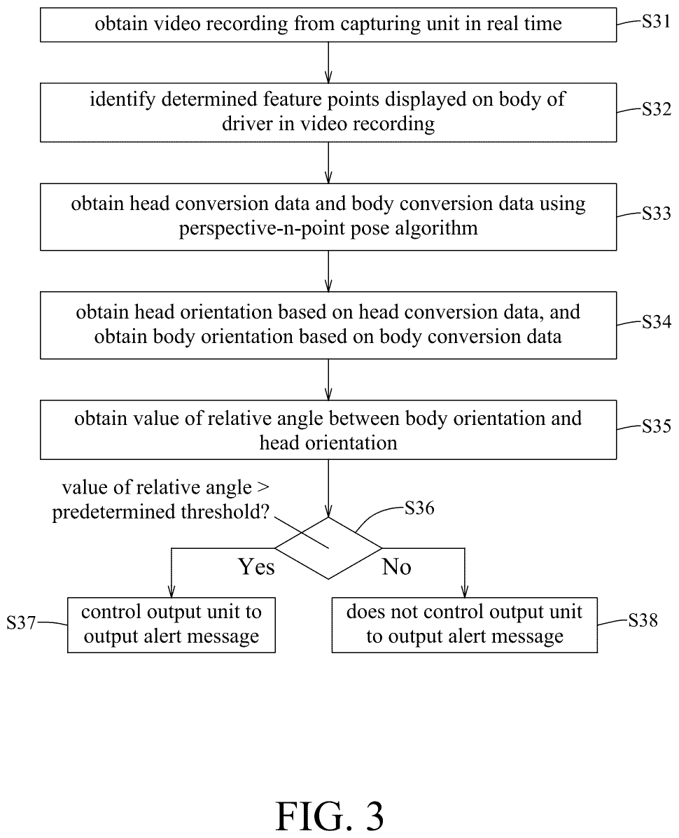

It should be noted that steps S31 to S38 and the flow chart shown in FIG. 3 merely constitute one example of the method of the disclosure, and steps S31 to S38 may be combined, divided, or switched in order as long as the method under such adjustment achieves substantially the same function in substantially the same way as provided in the embodiment. That is to say, the order of the steps of the method is not limited to the abovementioned example.

In summary, according to the disclosure, the system 1 is configured to obtain the relative angle between the body orientation and the head orientation, so as to more accurately determine whether the driver has turned his/her head for determining whether to output the alert message. As such, when the driver is driving the vehicle (especially large vehicles), the system 1 provided in this disclosure is able to reduce the occurrence of outputting the alert signal, which may distract the driver, when the driver is actually performing normal driving actions (e.g., turning head or body to check the surroundings).

In the description above, for the purposes of explanation, numerous specific details have been set forth in order to provide a thorough understanding of the embodiment(s). It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects; such does not mean that every one of these features needs to be practiced with the presence of all the other features. In other words, in any described embodiment, when implementation of one or more features or specific details does not affect implementation of another one or more features or specific details, said one or more features may be singled out and practiced alone without said another one or more features or specific details. It should be further noted that one or more features or specific details from one embodiment may be practiced together with one or more features or specific details from another embodiment, where appropriate, in the practice of the disclosure.

While the disclosure has been described in connection with what is (are) considered the exemplary embodiment(s), it is understood that this disclosure is not limited to the disclosed embodiment(s) but is intended to cover various arrangements included within the spirit and scope of the broadest interpretation so as to encompass all such modifications and equivalent arrangements.

What is claimed is:

1. A method for distraction alert to be implemented by a system disposed on a vehicle, the method comprising:
obtaining a video recording that contains a part of a human body in real time, where the part of the human body includes a body portion and a head portion;
obtaining a body orientation of the body portion and a head orientation of the head portion based on the video recording;
comparing the body orientation and the head orientation, so as to obtain a value of a relative angle between the body orientation and the head orientation;
determining whether the value of the relative angle is greater than a predetermined threshold; and
in response to determining that the value of the relative angle is greater than the predetermined threshold, outputting an alert message.

2. The method as claimed in claim 1, wherein obtaining the body orientation includes obtaining a plurality of determined feature points that are on the part of the human body based on the video recording, and then obtaining the body orientation based on the plurality of determined feature points.

3. The method as claimed in claim 2, wherein the plurality of determined feature points include two determined shoulder feature points that are respectively located at two shoulder areas of the body portion, and a plurality of determined head feature points that are located at the head portion.

4. The method as claimed in claim 3, the system storing 3-dimensional (3D) human model data that includes a plurality of reference feature points which respectively correspond to a plurality of reference coordinate sets, wherein the plurality of determined feature points are respectively associated with the plurality of reference feature points, and respectively correspond to a plurality of determined coordinate sets, wherein obtaining the body orientation includes:
obtaining the plurality of determined feature points from the video recording;
obtaining, using a perspective-n-point pose algorithm, conversion data that represents a coordinate conversion relationship between the plurality of determined coordinate sets and the plurality of reference coordinate sets; and
obtaining the body orientation based on the conversion data.

5. The method as claimed in claim 1, wherein obtaining the head orientation includes obtaining a plurality of determined feature points that are on the part of the human body based on the video recording, and then obtaining the head orientation based on the plurality of determined feature points.

6. The method as claimed in claim 5, wherein the plurality of determined feature points are located at the head portion.

7. The method as claimed in claim 6, the system storing 3-dimensional (3D) human model data that includes a plurality of reference head feature points which respectively correspond to a plurality of reference head coordinate sets, wherein the plurality of determined feature points are respectively associated with the plurality of reference head feature points, and respectively correspond to a plurality of determined head coordinate sets, wherein obtaining the head orientation includes:
obtaining a plurality of determined feature points from the video recording;
obtaining, using a perspective-n-point pose algorithm, conversion data that represents a coordinate conversion relationship between the plurality of determined head coordinate sets and the plurality of reference head coordinate sets; and
obtaining the head orientation based on the conversion data.

8. The method as claimed in claim 1, further comprising:
obtaining location data of the vehicle;
determining whether the location data indicates that the vehicle is located in a pausing area; and
in response to determining that the vehicle is located in the pausing area, not outputting the alert message.

9. A system for distraction alert adapted to be disposed in a vehicle, comprising:
a processing unit; and
an output unit electrically connected to said processing unit;
wherein said processing unit is configured to
obtain a video recording that contains a part of a human body in real time, where the part of the human body includes a body portion and a head portion,
obtain a body orientation of the body portion and a head orientation of the head portion based on the video recording,
compare the body orientation and the head orientation, so as to obtain a value of a relative angle between the body orientation and the head orientation,
determine whether the value of the relative angle is greater than a predetermined threshold, and
in response to determining that the value of the relative angle is greater than the predetermined threshold, control said output unit to output an alert message.

10. The system as claimed in claim 9, wherein said processing unit is configured to obtain a plurality of determined feature points that are on the part of the human body based on the video recording, and then obtain the body orientation based on the plurality of determined feature points.

11. The system as claimed in claim 10, wherein the plurality of determined feature points include two determined shoulder feature points that are respectively located at two shoulder areas of the body portion, and a plurality of determined head feature points that are located at the head portion.

12. The system as claimed in claim 11, further comprising a storage medium electrically connected to said processing unit and storing 3-dimensional (3D) human model data that includes a plurality of reference feature points which respectively correspond to a plurality of reference coordinate sets, wherein the plurality of determined feature points are respectively associated with the plurality of reference feature points, and respectively correspond to a plurality of determined coordinate sets, wherein said processing unit is configured to:
obtain the plurality of determined feature points from the video recording;
obtain, using a perspective-n-point pose algorithm, conversion data that represents a coordinate conversion relationship between the plurality of determined coordinate sets and the plurality of reference coordinate sets; and
obtain the body orientation based on the conversion data.

13. The system as claimed in claim 9, wherein said processing unit is configured to obtain a plurality of determined feature points that are on the part of the human body based on the video recording, and then obtain the head orientation based on the plurality of determined feature points.

14. The system as claimed in claim 13, wherein the plurality of determined feature points are located at the head portion.

15. The system as claimed in claim 14, further comprising a storage medium electrically connected to said processing unit and storing 3-dimensional (3D) human model data that includes a plurality of reference head feature points which respectively correspond to a plurality of reference head coordinate sets, wherein the plurality of determined feature points are respectively associated with the plurality of reference head feature points, and respectively correspond to a plurality of determined head coordinate sets, wherein said processing unit is configured to:
obtain a plurality of determined feature points from the video recording;
obtain, using a perspective-n-point pose algorithm, conversion data that represents a coordinate conversion relationship between the plurality of determined head coordinate sets and the plurality of reference head coordinate sets; and
obtain the head orientation based on the conversion data.

16. The system as claimed in claim 9, further comprising a positioning unit configured to obtain location data of the vehicle, wherein said processing unit is configured to:
determine whether the location data indicates that the vehicle is located in a pausing area; and
in response to determining that the vehicle is located in the pausing area, generate and output a pausing notification to said output unit for said output unit to not output the alert message.
